(19) United States
(12) Patent Application Publication
(10) Pub. No.: US 2007/0218150 A1
(43) Pub. Date: Sep. 20, 2007

(54) Active Oxygen Scavenging Agent and Moisturizing Agent Containing Wild Watermelon Extract

(76) Inventors: Kinya Akashi, Nara (JP); Akiho Yokota, Nara (JP)

Correspondence Address: POSZ LAW GROUP, PLC, 12040 SOUTH LAKES DRIVE, SUITE 101, RESTON, VA, 20191, US

(21) Appl. No.: 11/579,183

(22) PCT Filed: Apr. 26, 2005
(86) PCT No.: PCT/JP05/07909

§ 371 Date: Oct. 30, 2006

(30) Foreign Application Priority Data
Apr. 30, 2004 (JP) 2004-135241

(51) Int. Cl. A61K 36/00; A23L 1/30; A61P 17/14; A61P 39/06; A61P 17/16; A23L 2/52

(52) U.S. Cl. 424/758; 424/725

(57) ABSTRACT

It is intended to provide a composition containing wild watermelon extract. More specifically speaking, it is intended to provide an active oxygen scavenging agent containing wild watermelon extract, a method of producing the same and a method of using the same, and a moisturizing agent containing wild watermelon extract, a method of producing the same and a method of using the same. Attention is paid to the facts that wild watermelon juice per se employed as an active oxygen eliminator shows an activity of eliminating active oxygen about 10 times higher citrulline, which is contained in wild watermelon juice, at the same concentration and that wild watermelon extract has not only the ability to eliminate active oxygen but also a favorable moisturizing effect. Thus, there have been developed an active oxygen eliminator and a moisturizing agent as well as foods, cosmetics, bathing preparations, hair care products, drugs and quasi drugs containing extract of wild watermelon being a native of Kalahari Desert in Africa.

### TECHNICAL FIELD

The present invention relates to the active oxygen scavenging agent to scavenge active oxygen, moisturizing agent to moisturize, foods, antioxidant food additives, health foods, cosmetics, hair care products, bathwater agents and hygiene products containing these.

### BACKGROUND ART

The active oxygen such as hydroxyl radical, etc., is a main cause in triggering various diseases and aging, moreover causing skin problems such as freckles and age spots. The addition of antioxidant substances in foods or cosmetics is wide spread and in such cases, mainly a chemically synthesized artificial compound is often used. But these groups of antioxidant substances often have had side-effects on the human body, or have problems of severe light aging and auto-oxidation. Therefore, it has been desired to develop a naturally-derived antioxidant with excellent degradation activity of the active oxygen and with a high degree of safety.

Conventionally, as naturally-derived antioxidants, citrulline is known, for example, and there exists an invention of an active oxygen scavenging agent and cosmetic compositions containing citrulline (Patent Literature 1). Citrulline has excellent active oxygen-scavenging ability and wild watermelon native to Botswana contains a significant quantity of citrulline. The active oxygen-scavenging ability of wild watermelons was considered to derive from citrulline. However, as its addition quantity could be less if there is a more active one than the active oxygen-scavenging agent containing citrulline, development of a composition having higher activity of active oxygen scavenging ability had been expected.

Moreover, as the extract of cultivated watermelons contains sucrose, it could not be directly used for cosmetics. So, the raw materials of watermelon to make juice having low sucrose content have been desired.

Moreover, it has been desired to develop cosmetics, food additives, bathwater agents and hair care products having a high degree of safety and a good shelf life and effectively protecting the human body from toxic light and toxic oxygen.

Patent Literature 1: Japanese Published Unexamined Patent Application No. 2002-226370

### DISCLOSURE OF THE INVENTION

### Problems to be Solved by the Invention

The present invention provides compositions containing the extract of wild watermelon. More specifically, it provides an active oxygen scavenging agent containing the extract of an wild watermelon, the manufacturing process, use method, and the moisturizing agent containing the extract of wildwatermelon, its manufacturing process and use method.

Means for Solving the Problems

The inventors have found that there is a strong active oxygen scavenger activity in wild watermelon of Botswana, as a result of devoting themselves to research to solve the problem.

This wild watermelon accumulates a significant amount of citrulline by drying stress, etc. As it is known that citrulline has active oxygen scavenging ability (Patent Literature 1), it has been thought that the active oxygen scavenging ability of wild watermelon derives from citrulline. However, the inventors measured the active oxygen scavenging activity of the juice of wild watermelon itself and showed active oxygen scavenging activity about ten times higher compared to the corresponding concentration of the citrulline aqueous solution. Consequently, it became clear that active oxygen scavenging activity of wild watermelon juice depends on other components more than citrulline. Namely, it was found that the activity of active oxygen scavenging is 10 times higher in the case which wild watermelon juice was used itself as the active oxygen scavenging agent than in the case in which a corresponding concentration of citrulline which is contained in wild watermelon juice was used. In addition, inventors have found that the wild watermelon extract has a rich moisturizing effect with the active oxygen scavenging ability and completed the invention.

The present invention provides the following inventions.

From the first standpoint of the present invention, an active oxygen scavenging agent containing wild watermelon extract is provided. According to the present invention, a novel active oxygen scavenging agent can be obtained.

From the second standpoint of the present invention, a method to scavenge active oxygen by wild watermelon extract is provided. According to the present invention, a novel use of wild watermelon extract can be provided.

From the third standpoint of the present invention, an active oxygen scavenging agent in which wild watermelon extract is an active ingredient is provided. According to the present invention, a novel active oxygen scavenging agent in which wild watermelon extract is the active ingredient can be obtained.

From the fourth standpoint of the present invention, a composition containing the active oxygen scavenging agent according to the first or the third standpoint is provided. According to the present invention, a new composition with the active oxygen scavenging ability can be obtained.

From the fifth standpoint of the present invention, a moisturizing agent containing wild watermelon extract is provided. According to the present invention, a novel moisturizing agent is provided.

From the sixth standpoint of the present invention, a method to moisturize by using wild watermelon extract is provided. According to the present invention, a novel method to moisturize with the wild watermelon extract is provided.

From the seventh standpoint of the present invention, a composition containing the moisturizing agent according to the fifth standpoint is provided. According to the present invention a novel composition is provided.

From the eighth standpoint of the present invention, a cosmetic containing the moisturizing agent according to the above fifth standpoint is provided. According to the present invention, a new cosmetic is provided.

From the ninth standpoint of the present invention, a composition being wild watermelon extract, having twice or higher the antioxidant activity compared to citrulline aqueous solution of a corresponding concentration is provided. According to the present invention, the composition with a higher antioxidant activity than citrulline aqueous solution used as a single agent can be obtained.

From the tenth standpoint of the present invention, a method to manufacture the active oxygen scavenging agent or moisturizing agent characterized by using wild watermelon extract as a whole or part of the raw material is provided. According to the present invention, the method for producing a novel active oxygen scavenging agent or moisturizing agent can be obtained.

From the eleventh standpoint of the present invention, a composition having a moisturizing effect derived from wild watermelon extract is provided. According to the present invention, the composition having a novel moisturizing effect can be obtained.

From the twelfth standpoint of the present invention, fruit juice drink characterized by containing a part of wild watermelon extract is provided. According to the present invention, a novel fruit juice drink containing the wild watermelon extract is provided.

From the thirteenth standpoint of the present invention, a bathwater agent containing the moisturizing agent according to the fifth standpoint is provided. According to this invention, a novel bathwater agent is obtained.

From the fourteenth standpoint of the present invention, a shampoo or a conditioner containing the moisturizing agent according to the fifth standpoint is provided. According to the present invention, a novel moisturizing shampoo or hair conditioner is provided.

From the fifteenth standpoint of the present invention, a hair growth tonic containing the moisturizing agent according to the fifth standpoint is provided. According to the present invention, a novel moisturizing hair growth tonic is provided.

Effects of the Invention

According to the present invention, the active oxygen scavenging agent with high active oxygen scavenging activity, moisture rich moisturizing agent and composition containing these, etc., are provided.

### DESCRIPTION OF THE PREFERRED EMBODIMENTS

Hereinafter, examples of the present invention are described but the illustrative embodiment is not limited to these.

In the present invention ‘wild watermelon’ means wild watermelon (Citrullus lanatus) native to Bostwana which grows naturally in the Kalahari Desert, in Africa and the available watermelon in the present invention among the progeny watermelon varieties by crossbreeding of these, including one which grows artificially in a field, etc. ‘The available watermelon in the present invention’ means that the extract of watermelon flesh has more than twice or higher the scavenging activity of active oxygen than a single citrulline aqueous solution of the same concentration as the citrulline concentration of the extract of such watermelon, and that the total of each concentration of fructose, glucose and sucrose is 4% or less (weight/weight percent. Same as above). More preferably, it means that the extract of watermelon flesh has more than five times higher scavenging activity of the active oxygen than a single citrulline aqueous solution of the same concentration as the citrulline concentration of the extract of watermelon and that the total of each concentration of fructose, glucose and sucrose is 2% by weight or less. As Bushmen have eaten wild watermelons native to Botswana for a long time, its safety as a food is considered to not be an issue.

It is reported that the content rate of sugar in the flesh of the cultivated variety of watermelon grown in Japan, etc., is 5.03% fructose, 1.57% glucose, and 0.98% sucrose (http://www.suika-net.co.jp/nutri.html), while the content rate of sugar in the flesh of wild watermelon native to Botswana is 0.88% fructose, 0.45% glucose, and 0.05% or less of sucrose. As the wild watermelon native to Botswana have a low sugar concentration and are not sweet, they can be used across very wide applications. In particular, with respect to foods and drinks, it is possible to add to any food or drink.

In this specification, ‘the wildwatermelon extract’ means that which is extracted from wild watermelon, including that which is ground up as wildwatermelon fruits, juice of wildwatermelon fruit, filtrate which filters that which is ground up as wild watermelon fruits or supernatant of the centrifugation of it. Moreover, it contains the extract which is extracted using a polar or nonpolar solvent, etc. The extraction solvent and the extraction method are not particularly limited, and any solvent or any method may be used as long as it is an extract of components derived from wild watermelon. The part for extraction is not particularly limited, but it is preferable to use flesh, especially preferable to use the flesh part whose hull, endothelium and seeds are removed.

An obtained extract may be used without modification, or in concentrated or dried form. And, a concentrated or dried one may be used to be dissolved or suspended in the appropriate solvent. It may be used by obtaining a fraction having active oxygen scavenging activity using liquid chromatography, etc., from the extract.

The active oxygen scavenging agent of the present invention contains the wild watermelon extract. More preferably, it contains the wild watermelon extract as the active ingredient to scavenge active oxygen. The content of the wild watermelon extract in the active oxygen scavenging agent can be changed according to its application.

The active oxygen scavenging agent of the present invention can be used in order to prevent oxidation of an ingredient included in quasi drugs, drug medicines, cosmetics, foods and drinks, feedstuff, bathwater agents, hair care products, etc. With regard to additional amounts, an amount may be added which can prevent oxidation of an ingredient included in quasi drugs, drug medicines, cosmetics, foods and drinks, feedstuff, bathwater agents, hair care products etc.

As the composition containing the active oxygen scavenging agent of the present invention, for example, quasi drugs, drug medicines, cosmetics, foods and drinks, feedstuff, bathwater agents and hair care products, etc., can be mentioned. These various compositions-are not particularly limited if adversely affected by oxidation. Herein, ‘foods and drinks’ mean things that can be used for drinks and foods.

As foods and drinks, for example, oils and fats, foods and drinks containing oils and fats, and foods and drinks containing dyes (especially natural dye), etc., can be mentioned.

Oils and fats include, for example, oils and fats of plants and animals such as corn oil, canola oil, cotton oil, soybean oil, safflower oil, sunflower oil, sesame oil, wheat germ oil, olive oil, evening primrose oil, camellia oil, tea oil, avocado oil, ricinus, coffee oil, cashewnut oil, cacaobeanoil, peanut oil, fish oil, palm oil, palm kernel oil, lard, beef fat and chicken fat, etc., and the partly hydrogenated oil of the above oil and fat of plants and animals; unsaturated fatty acids such as oleic acid, linoleic acid, alpha-linoleic acid, gamma-linoleic acid, eicosapentaenoic acid, docosahexaenoic acid, arachidonic acid and mead acid, etc., and esters or unsaturated alcohols of these; oiled food such as butter, margarine, shortenings and dressings and soon can be mentioned.

Moreover, as foods and drinks containing oils and fats, for example, foods containing relatively significant amounts of oils such as doughnuts, fried tofu, fried confectionery, chocolate and instant noodles, etc., can be mentioned.

In addition, the following are quoted as subject foods and drinks, for example, Japanese confectionery such as rice crackers, Japanese crackers, millet-and-rice cake, buns with bean-jam filling, candy; various western confectioneries such as cookies, biscuits, crackers, pies, sponge biscuits, sponge cakes, doughnuts, waffles, puddings, buttercream, pastrycream, cream puffs, chocolate, chocolate confectionery, candy, chewing gum, gummy bears, jelly, pan cakes, breads, etc.; snacks such as potato chips, water ice such as ice cream, ice candy, sherbet; soft drinks such as lactic acid beverages, lactic acid bacteria beverages, condensed dairy drinks, fruit juice, drinks without fruit juice, juice with pulp, functional beverages, clear carbonated beverages, carbonated beverages with fruit juice, fruit colored carbonated beverages and the like; favorite beverages such as green tea, tea, instant coffee, cocoa, canned coffee, professional-use coffee, and the like; milk products such as fermented milk, processed milk, cheese, and the like; soybean processed foods, alcoholic beverages such as low-malt beer, wine, Japanese sake, shochu, shochu-based beverages, liqueurs, brandy, whiskey, and the like; marmalade, jams, fruits preserved in syrup; pastes such as flower paste, peanut paste, fruit paste and the like; pickles; meat products such as ham, sausage, bacon, dry sausage, beef jerky, etc.; fish and seafood products and the salted dried fish such as fish ham, fish meat sausage, steamedfishpaste, fishsausage, mincedflesh, tempura, and the like; various dried fish such as dried bonito, dried mackerel, dried pompano; dried small sardine, fermented products of sea urchins and squid; various seafood delicacies such as dried cuttlefish, dried fish in sweet cooking rice wine, dried shellfish, dried codfish, lox and the like; foods boiled in soy sauce made from layer, small fry, shellfish, dried cuttlefish, edible wild plants, mushrooms, kelp, and the like; curries such as instant curry, retort curry, canned curry and the like; various condiments such as miso, miso powder, soy sauce, soy sauce powder, unrefined soy sauce, fish sauce, sauce, ketchup, mayonnaise, bouilloncubes, oystersauce, grilled meat sauce, curry roux, stew roux, soup stock cube, instant bouillon and the like; various foods and drinks such as microwave meals and frozen foods containing fat and oil. In addition, each of the above foods and drinks is an example of those which the active oxygen scavenging agent of the present invention is applied to, and is not in any way limited to these foods and drinks.

As drug medicines, quasi drugs and cosmetics, troche, liver oil drops, mouthwash, toothpaste, mouth fresheners, bad breath inhibitors, sunscreens, skin lotions, various barrier creams, foundations and lipsticks, etc., are mentionedandas feedstuff, for example, various cat foods, dog foods, fish food for ornamental fish and fish food for farm-raised fish, etc., are mentioned. Moreover, it includes bathwater agents, hair care products, forexample, shampoos, hairconditioners, hairgrowth tonics and the like.

In addition, the active oxygen scavenging agent of the present invention can be used in combination with other active oxygen scavenging agents, for example, tocopherol, L-ascorbic acid, sodium erythorbate, BHA, sesamin, astaxanthin and the like.

The active oxygen scavenging agent of the present invention contains wild watermelon extract. The blending quantity of the wild watermelon extract contained in the active oxygen scavenging agent can be changed according to its use and its object as well other active oxygen scavenging agents and is not particularly limited. And, the wild watermelon extract itself can be used as anactive oxygen scavenging agent directly.

The active oxygen scavenging agent of the present invention can be used for improving or protecting the various disorders caused by active oxygen in vivo, adding an effective dose for active oxygen scavenging action to an oral composition such as foods, drug medicines, etc., and to skin drugs for external use such as cosmetics and external medicines, etc.

In addition, as various disorders caused by active oxygen in vivo, various inflammations (including periodontal diseases such as gingivitis, periodontitis and, pneumonia, etc.), cardiovascular diseases such as cardiac infarction, arterial sclerosis etc., diabetes, diabetic complications such as cataracts, etc., various cancers (for example, including lung cancer, stomach cancer, etc., caused by an initiator and promoter), apoplexy, stiff neck, excessive sensitivity to cold, hypertension, senile dementia, dementia by Alzheimer type by a toxicity of the β-amyloid protein, disorders such as aging, etc., (disorders caused by an active oxygen free radical) and skin lesions such as freckles, age spots, wrinkles, etc., are knownbut the present invention covers disorder, etc., including not only the above disorders and skin disorders that are hitherto known to have a causal relationship with active oxygen but also those whose causal relationship to active oxygen will become clear in the future.

Moreover, the effective dose for the active oxygen scavenging function means the dose which exerts an active oxygen scavenging function, or which can lower or eliminate active oxygen in vivo. The effective dose for active oxygen scavenging function varies according to objects, application parts, etc., and is not particularly limited as long as it exerts the above effect, but more specifically, can exemplify the range of ordinarily 0.025˜1.00% (hereinafter referred to as % by weight), more preferably, 0.05˜99%, especially preferably 1%˜99% with an example as a proportion of the containing amount of the botanical extract (equivalent in dry substances) of various compositions such as foods and drinks, drug medicines, cosmetics, etc.

For example, if it is used as raw materials of cold drinks such as juice, etc., it can be used in the range of 1˜99%. In this case, it may contain flesh.

In the case of use as a health food, concentrated wild watermelon juice can be used in the form of tablets, powders, capsules, etc.

In addition, the active oxygen scavenging agent of the present invention can be used together with heretofore known any various components (for example, extending agents, vehicles, lubricants, binders, oil solutions, moisturizing agents, thickeners, antiseptic agents, emulsifying agents, chelate agents, colorants, pH adjusters, medicinal properties, ultraviolet absorbers and aroma chemicals, etc.) depending on the dispensing object, and herewith can be prepared in any form such as various foods (for example, health foods, etc.), oral administration agents, drugs for external use (for example, creams, milk lotions, lotions, gel agents, ointments andpowders etc.), cosmetics (for example, foundations, pack, essences, lip creams, lipstick sun care products) etc.

In addition, the active oxygen scavenging agent of the present invention can be used in combination with other active oxygen scavenging agents.

Moreover, the present invention also provides a moisturizing agent containing the wild watermelon extract. The wild watermelon extract, as shown in Example 2, has rich moisture comparable to glycerin. Therefore, it can also be used as a moisturizing agent. The moisturizing agent of the present invention (hereinafter called the agent) is not particularly limited, and can be used as a moisturizing agent for skin (including the face, limbs (pertaining to elbows, knees and heels), body, scalp) hair and nails, etc., for example. The moisturizing agent of the present invention may substantially consist of only wild watermelon extract or may be mixed with other components. As other components, if it can be dispensed as external medicine for skin (pertaining to drug medicines, cosmetics, fragrances, cleaning agents, bathwater agent, hair care products and other quasi drugs), various components can be used according to application site, use and intended purpose.

For example, other moisturizing agents, external bases, fragrances, thickeners, preservative agents, oil content, fatty acids, sterols, surface acting agents, water-soluble multiple alcohols, powders, silicones and other components, etc. Adding appropriately these components according to use and intended purpose, it can easily make the applicable form such as liquid form, solid form, paste form, gel form and emulsifying form etc.

Moreover, if it is a material of cosmetics, raw materials can be dispensed such as fats and oils, waxes, carbon hydrides, fatty acids, alcohols, esters, surface acting agents, whitening agents and moisturizing agents, etc., which are used ordinarily for cosmetics, etc., within the range without losing the moisturizing action. Concentration of the wild watermelon extract in the agent can be determined according to use and purpose of the agent.

In addition, the agent may be used for applying to the application part such as skin, hair and nails, etc., and can be used for combining external medicines for skin such as drug medicines and toiletries, etc. Therefore, the present invention provides a composition containing the above moisturizing agent of the present invention (the agent). The agent preferably contains wild watermelon extract as an active component. While the composition containing the agent is not particularly limited, it can be illustrated by examples, (1) cosmetics for skin containing creams, milky lotions, water lotions, pack agents, foundation (foundation creams), eye masks, ice creams, lipsticks and chapsticks, etc., (2) cosmetics for hair containing hair gels (for hair), hair sprays, hair liquids, hairtonics, hairmousse, pomade, stick pomade, rinses and hair treatments etc., (3) washing agents containing hair shampoos, body-wash, hand soaps and soaps (including both liquids and solids), etc., (4) bathwater agents (5) cosmetics for nails including nutrient creams for nails, manicures, (containing nail coats) and polish removers, etc., (6) hair growth tonic, etc. The composition containing moisturizing agents of the present invention can prevent the surface of the skin, etc., from drying out and exert a moisturizing effect by keeping or adhering the wild watermelon extract to the surface such as skin, etc., as a moisturizing agent.

The amount of a moisturizing agent mixed in a composition containing a moisturizing agent of the present invention can be determined appropriately according to the kind of composition, use and purpose, etc., and is not particularly limited. For example, the concentration (concentration is % by weight) is 0.1%˜100%, preferably 1%˜100%, more preferably 1%˜70%, more preferably 1%˜50%, especially preferably 3%˜30%. In addition, while the moisturizing agent of the present invention contains the extract of wild watermelon, the possibility of causing environmental pollution is not likely as such an extract is naturally-derived, even if it flows out to the natural world.

Moreover, a moisturizing agent of the present invention may be contained in drug medicines, foods and drinks, and feedstuff, etc. While applicable drug medicines, foods and drinks, and feedstuff, etc., are the same as the active oxygen scavenging agent, the containing concentration can be changed according to use.

The extract of wild watermelon shown in the example, which contains citrulline 0.288 mM, had about ten times higher active oxygen scavenging activity than an aqueous solution containing only citrulline of the same concentration. That is, the activity of citrulline which is occupied in the active oxygen scavenging activity of wild watermelon extract is about one tenth, and was shown that the contribution of components other than citrulline was far more significant. By this discovery, an extract of wild watermelon, a composition having antioxidation activity more than twice as high as the corresponding concentration of citrulline can be provided. As a composition, equivalent quasi drugs, drug medicines, cosmetics, foods and drinks, feedstuff, bathwater agents and hair care products, etc., than a composition containing said active oxygen scavenging agent can be used.

Moreover, according to the present invention, the method to produce the active oxygen scavenging agent or moisturizing agent which is characterized by using the extract of wild watermelon as a part of the raw materials is provided. This can produce a novel active oxygen scavenging agent or moisturizing agent by blending or reacting the wild watermelon extract with other components. In this production method, it is similar to the production method of the active oxygen scavenging agent or moisturizing agent as is well known to those skilled in the art besides adding the extract of the wild watermelon as a raw material.

The present invention is explained as follows using examples, but it is not in any way limited to these examples.

### EXAMPLE 1

(Activity of Active Oxygen Scavenging)

Exodermises, endodermis, and seeds of wild watermelon native to Botswana were removed and the part of the flesh is obtained. After that, the flesh extract was obtained by crushing this flesh with a blender and by filtering it through gauze and Miracloth.

The active oxygen scavenging ability of the wild watermelon fruit extract is assayed by competitive reaction analysis using salicylic acid oxidation as an index. Specifically, 2 mM salicylic acid and the wild watermelon fruit extract of various concentrations are dissolved in 50 mM phosphate buffer (pH 7.4). After that, by adding 0.6 mM of hydrogen peroxide, 0.15 mM of FeEDTA and 0.26 mM of ascorbic acid, hydroxyl radical is generated, and reacted at 25 degrees for 90 minutes. Quantitation of dihydroxyl benzoic acid (DHBA) generated by salicylic acid oxidation followed the method as set forth in the literature (Smirnofff and Cumbes (1989) Phytochemistry, 28: 1057-1060.)

As a result, by containing 25% of the fruit extract, oxidation of salicylic acid is inhibited by as much as 92% compared to the control added with only water; this has revealed that the wild watermelon fruit extract has exceptional active oxygen degradation ability. On the other hand, while 25% of the wild watermelon extract contains about 50 mg/l (0.29 mM) of citrulline, 50 mg/l of citrulline solution was assayed for the active oxygen scavenging activity and salicylic acid oxidation was inhibited by only 6%. This shows a substance other than citrulline in the wild watermelon extract bears high active oxygen scavenging activity of the extract.

### EXAMPLE 2

(Active Oxygen Scavenging Activity)

The moisturizing property of the wild watermelon fruit extract native to Botswana was estimated. Each 100 μL of 5% aqueous solution of the wild watermelon extract and a comparative solution, 5% glycerol aqueous solution were dropped on the filter paper (Whatman filter paper 1, 42.5 mm, Whatman Ltd.), and was left standing at a temperature of 25° C. and at a relative humidity of 35%, and the water residual ratio was measured after two hours by weighing method. The result is shown in FIG. 2.

In the case of dropping only water, 27% of the water was left but the water residual ratio could be raised to 33% by using glycerin which is widely used in cosmetics as a moisturizer. Compared with this, using the wild watermelon extract, the water residual ratio is 35%, water loss from the filter paper was confirmed to be suppressed more than glycerin and it is elucidated that wild watermelon fruit extract has a high moisturizing effect.

### EXAMPLE 3

The present invention can also be used as a bathwater agent. In one of extracts of wild watermelon of the present invention, the fruit juice of the flesh part of watermelon native to Botswana was dried, and was powdered and was made as a bathwater agent. Dissolving 10 g of powder in about 200 L of hot water, ten trial subjects with dry skin were asked to use it for one month. As a control, another ten people were asked to use a commercial bathwater agent for one month in a similar way. As a result, seven of ten who used the bathwater agent of the present invention answered that the dry feeling of skin had improved. On the other hand, four of ten control subjects answered that the dry feeling of skin had improved. Therefore, the bathwater agent of the present invention is considered to have an effect to improve the dry feeling of skin.

### EXAMPLE 4

After filtering one of the extracts of wild watermelon of the present invention, the fruit juice of the flesh part of wild watermelon native to Botswana through gauze, diluting it with a pure water to become 10% fruit juice and 10% ethanol, a lotion was prepared by filtering it again. Ten trial subjects with dry skin used it for one month. Another ten people were asked to use a commercial lotion for one month in a similar way as a control. As a result, nine of ten who used the lotion of the present invention answered that the dry feeling of skin had improved. On the other hand, four of ten trial subjects as controls answered that the dry feeling of skin had improved. Therefore, the lotion of the present invention is considered to have an effect to improve the dry feeling of skin.

### EXAMPLE 5

Components of wild watermelon native to Botswana were analyzed by a high-performance liquid chromatography. An analysis was performed as follows. An analysis of fructose, glucose and sucrose were performed as follows. About 5 volumes of 50% ethanol (V/V) was added to 4˜5 g of crushed watermelon flesh, and sonication treatment was performed. A treatment condition was to use an ultrasonic generator (manufactured by Kokusai Electric Alpha Co., Ltd.) and to treat for half an hour. The volume of the sonicated sample was measured; it was filtered through a filter paper (Toyo Roshi, No. 5B). The filtrate was concentrated and dried by a rotatory evaporator and was dissolved in the appropriate volume of pure water. Filtering this solution through a membrane-filter (manufactured by Millipore Corporation, pore diameter 0.45 μL), it was analyzed by a high-performance liquid chromatography.

Analysis condition is as follows.
Model: LC-10ADvp (manufactured by Shimadzu Corporation)
Detector: Differential refractometer RID-10A (manufactured by Shimadzu Corporation)
Column: Wakosil 5NH2 Φ4.6 mm×250 mm (manufactured by Wako Pure Chemical Industries Ltd.)
Solvent: Acetonitrile 75%/pure water 25%
Flow rate: 1 mL/minute

As a result of analysis, the content of each sugar per 100 g of wild watermelon flesh native to Botswanaw as 0.88 g of fructose, 0.45 g of glucose and under a detectable level (0.05 g) of sucrose.

### INDUSTRIAL APPLICABILITY

The active oxygen scavenging agent and moisturizing agent of the present invention are available in industries of foods, cosmetics, drug medicines, quasi drugs, bathwater agents and hair care products, etc.

### BRIEF DESCRIPTION OF THE DRAWINGS

FIG. 1 shows an antioxidation effect (hydroxyl radical suppressing activity) of the wild watermelon extract native to Botswana. FIG. 1 shows, comparing active oxygen scavenging activity of extracts of wild watermelon with a single citrulline solution of its corresponding concentration, active oxygen scavenging activity of wild watermelon extract is about ten times higher than the single citrulline solution.

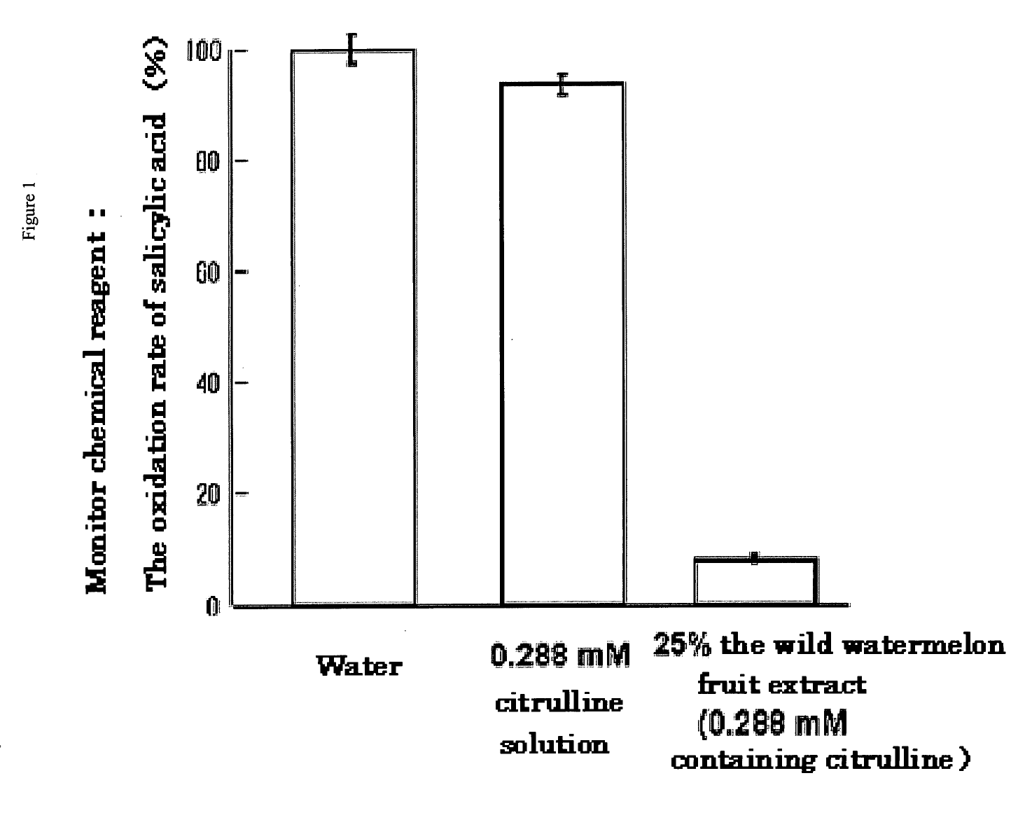

FIG. 2 shows the moisturizing effect of wild watermelon extract native to Botswana. FIG. 2 shows an effect comparable to glycerin, comparing the moisturizing effect of wild watermelon with that of glycerin,

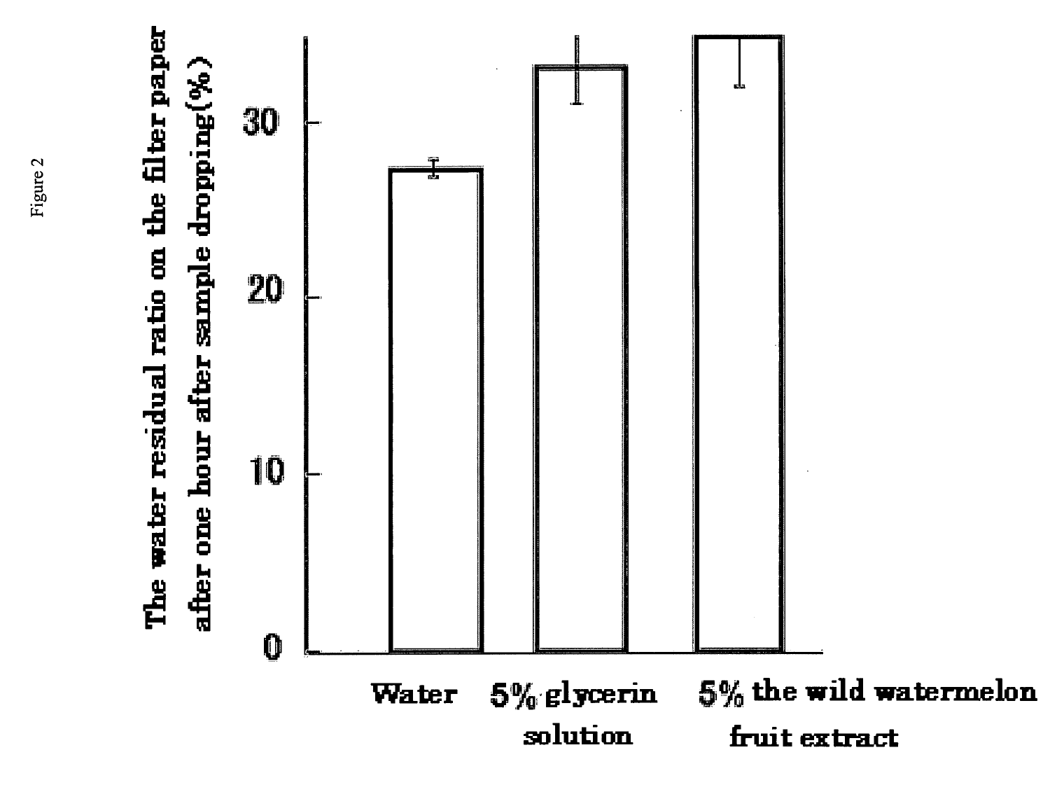

What is claimed is:

1. An active oxygen scavenging agent containing wild watermelon extract.

2. A method to scavenge active oxygen by wild watermelon extract.

3. An active oxygen scavenging agent in which wild watermelon extract is an active ingredient.

4. A composition containing the active oxygen scavenging agent according to claim 1 or 3.

5. A moisturizing agent containing wild watermelon extract

6. A method to moisturize by using wild watermelon extract

7. A composition containing the moisturizing agent according to claim 5.

8. A cosmetic containing the moisturizing agent according to claim 5.

9. A composition being wild watermelon extract, having twice or the higher antioxidant activity compared to citrulline aqueous solution of a corresponding concentration.

10. A method to manufacture the active oxygen scavenging agent or moisturizing agent by using wild watermelon extract as a whole or part of the raw material.

11. A composition having a moisturizing effect derived from wild watermelon extract.

12. Fruit juice drink characterized by containing a part of wild watermelon extract.

13. A bathwater agent containing the moisturizing agent according to claim 5.

14. A shampoo or a conditioner containing the moisturizing agent according to claim 5.

15. A hair growth tonic containing the moisturizing agent according to claim 5.
